(19) United States
(12) Patent Application Publication
(10) Pub. No.: US 2007/0123933 A1
(43) Pub. Date: May 31, 2007

(54) Manually operable equipment

(75) Inventors: Gregory W. Johnson, Milford, OH (US); John V. Hunt, Cincinnati, OH (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Ethicon Endo-Surgery, Inc.

(21) Appl. No.: 11/289,347

(22) Filed: Nov. 29, 2005

(51) Int. Cl. A61B 17/00

(52) U.S. Cl. 606/205

(57) ABSTRACT

Forceps for grasping tissue during a medical procedure. The forceps include a body, a jaw pivotally mounted on the body for movement between a grasping position and a releasing position, and a lever operatively connected to the jaw to drive the jaw from the grasping position to the releasing position. The forceps include a digit engagement member rotatably mounted on the lever. The digit engagement member has an engagement surface configured to engage a digit of an operator's hand during use. The engagement surface rotates relative to the lever as the lever drives the jaw from the releasing position to the grasping position

### BACKGROUND OF THE INVENTION

The present invention relates to a manually operable equipment, and more particularly to manually operable equipment having a digit engagement member that rotates independently from an actuation member.

Various tools and equipment such as medical devices, including laparoscopic forceps and conventional scissors, have actuation members which are moved to actuate the equipment. In many instances, the actuation member is a lever which is pivoted to actuate the equipment. As an example, FIG. 1 illustrates conventional laparoscopic forceps generally designated by the reference number 10. The forceps 10 have opposing levers 12, at least one of which is operatively connected to jaws 16 of the forceps. As the levers 12 move toward and away from each other, the jaws 16 close and open to grasp and release tissue or objects, respectively. Frequently, these levers 12 include rings 18 as shown in FIG. 1 for receiving a digit, such as a thumb or finger of a user. These rings 18 permit the user to selectively pivot the levers 12 in opposite directions to spread the levers such as when opening the jaws 14 and releasing tissue. As will be appreciated by those skilled in the art, without the rings 18 it is difficult to spread the levers 12 with one hand. Thus, the rings 18 enable one-handed operation of the equipment 10 which is particularly advantageous for medical equipment.

Digit engagement members such as rings 18 are well known in the art and generally function well for their intended purposes. However, when latex gloves are used in combination with equipment having digit rings, friction between the latex gloves and the digit rings can make the equipment more difficult to operate. In particular, the digit ring 18 tends to rotate around the user's digit as the user pivots the lever 12 when using the equipment. Friction between the glove and the surface of the digit ring 18 resists this rotation, hampering free movement of the equipment and user's hand, and sometimes causing the latex to pinch the user's digit or causing the latex to tear, thereby reducing its protective barrier characteristics. Accordingly, there is a need for equipment having digit rings, which eliminate or reduce the frictional effects that occur between the digit ring and the user's glove or hand, and which overcome the resulting disadvantages noted above, as well as other disadvantages which are known and appreciated by persons skilled in the art.

### BRIEF SUMMARY OF THE INVENTION

The present invention relates to forceps for grasping tissue during a medical procedure. The forceps comprise a body, a jaw pivotally mounted on the body for movement between a grasping position and a releasing position, and a lever operatively connected to the jaw to drive the jaw from the grasping position to the releasing position. In addition, the forceps include a digit engagement member rotatably mounted on the lever. The digit engagement member has an engagement surface configured to engage a digit of an operator's hand during use. The engagement surface rotates relative to the lever as the lever drives the jaw from the releasing position to the grasping position.

In another aspect, the present invention includes a equipment manually operable by motion of a digit of an operator's hand. The equipment comprises a body, an actuation member mounted on the body for movement between a first position and a second position, and a digit engagement member rotatably mounted on the actuation member. The digit engagement member has an engagement surface configured to engage a digit of an operator's hand during use. The engagement surface rotates relative to the actuation as the actuation member moves between the first position and the second position.

In yet another aspect, the present invention includes equipment manually operable by motion of a digit of an operator's hand. The equipment comprises a body, an actuation member mounted on the body for movement between a first position and a second position, and a digit engagement member mounted on the actuation member. The digit engagement member has an aperture defined by a series of flexible leaves spaced around the aperture. Each of the leaves has a tip configured to engage a digit of an operator's hand during use. The series of leaves deform to conform to the digit of the operator's hand.

Other aspects of the present invention will be in part apparent and in part pointed out hereinafter.

### BRIEF DESCRIPTION OF THE DRAWINGS

FIG. 1 is a side elevation of exemplary conventional equipment having digit rings for actuation;

FIG. 2 is a side elevation of a portion of equipment of a first embodiment of the present invention;

FIG. 3 is a separated perspective of the portion of equipment illustrated in FIG. 2;

FIG. 4 is a side elevation of a portion of equipment of a second embodiment of the present invention; and

FIG. 5 is a separated perspective of the portion of equipment illustrated in FIG. 4.

Corresponding reference characters indicate corresponding parts throughout the several views of the drawings.

### DETAILED DESCRIPTION OF THE INVENTION

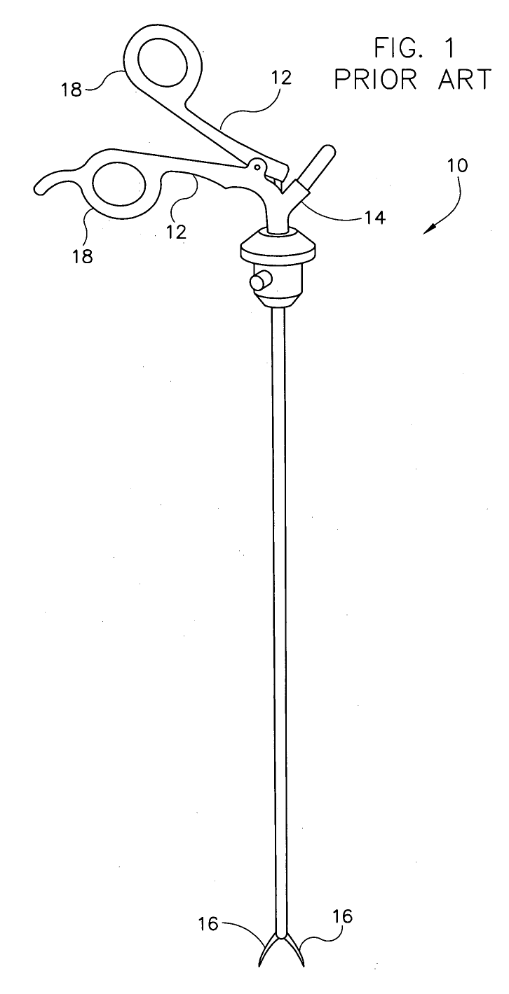

Referring now to the figures, and more particularly to FIG. 1, exemplary conventional equipment is designated in its entirety by the reference 10. The equipment 10, which in one embodiment consists of a pair of laparoscopic forceps, includes one or more levers 12. At least one of the levers 12 moves relative to a body 14 of the equipment 10. In the case of the exemplary embodiment, in which the equipment 10 is a pair of laparoscopic forceps, one lever 12 pivots relative to the body 14 to move jaws 16, which open and close in response to the levers moving away from each other and toward each other, respectively. At least one of said levers 12 includes a digit ring 18. The operator inserts his or her digits into corresponding digit rings 18 and moves his or her digits together to move the levers and actuate the equipment 10. As this equipment 10 is conventional, it will not be described in further detail.

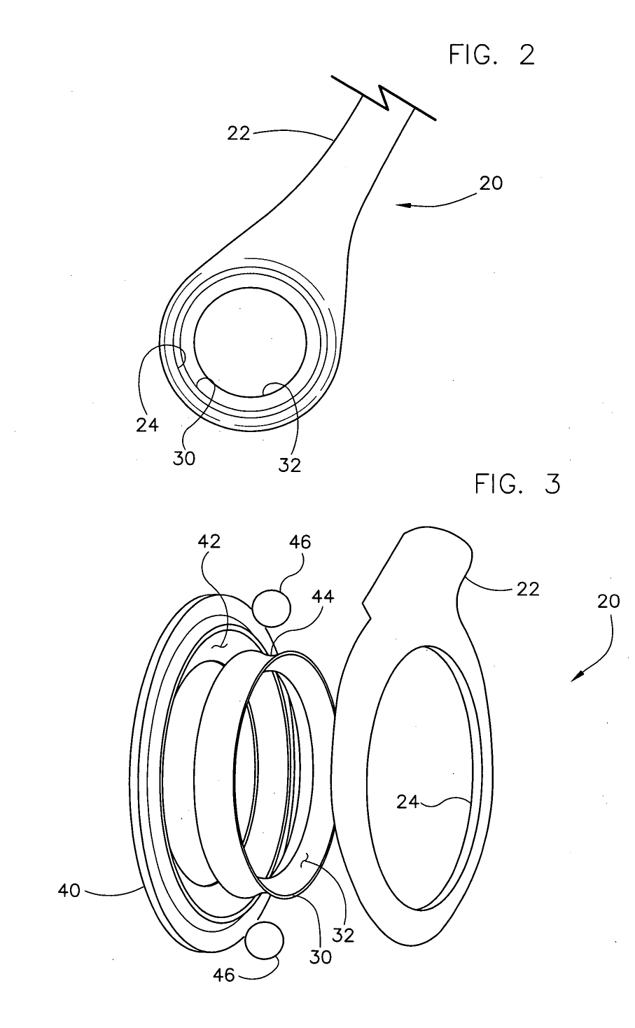

FIG. 2 illustrates a portion of equipment of a first embodiment of the present invention generally designated by the reference 20. The equipment 20 may be laparoscopic forceps similar to those described above but with additional and novel features as described below. The equipment 20 includes at least one actuation member 22 adapted for movement between a first position and a second position. The actuation member 22 is moveably connected to a body (e.g., a body similar to the body 14 of the equipment 10). In some exemplary embodiments, the actuation member 22 is a lever which pivots relative to the body. The actuation member 22 includes an opening 24. In some embodiments, the opening 24 is positioned at an end of the actuation member 22. A digit engagement member 30 is rotatably mounted on the actuation member 22 inside the opening 24. The digit engagement member 30 has an engagement surface 32 configured to engage a digit of an operator's hand during use. Although the digit engagement member 30 may have other shapes without departing from the scope of the present invention, in one embodiment the digit engagement member is a full annular 360° ring as shown so the digit engagement surface 32 defines an aperture sized and shaped for receiving a digit of the user. Although the opening 24 in the actuation member 22 may have other shapes without departing from the scope of the present invention, in one embodiment the opening is a fully enclosed circular hole as shown. The digit engagement surface 32 rotates relative to the actuation member 22 as the actuation member moves.

As shown in FIG. 3, the digit engagement member 30 is held in position by a separate retainer portion 40 fastened to the actuation member 22 adjacent the opening 24. Both the retainer portion 40 and the actuation member 22 include a shaped groove (although only the groove in the retainer portion is visible in FIG. 3), which collectively form an outer bearing race 42. The digit engagement member 30 also includes a groove forming an inner bearing race 44 having a shape which complements the outer bearing race 42 formed by the retainer portion 40 and the actuation member 22. The outer and inner bearing races 42, 44, respectively, receive a plurality of ball bearings 46 (only two of which are shown in FIG. 3) forming a bearing connecting the digit engagement member 30 and the actuation member 22 so the digit engagement member freely rotates relative to the actuation member. As will be appreciated by those skilled in the art, the components described above may be formed with other types of bearings connecting the digit engagement member 30 and the actuation member 22. For example, the ball bearings 46 may be replaced with roller or needle bearings (not shown) without departing from the scope of the present invention. Further, the bearings may be replaced with a low friction bushing which acts as a bearing to allow the digit engagement member 30 to freely rotate relative to the actuation member 22. Although the actuation member 22, the digit engagement member 30 and the retainer portion 40 may be made of other suitable conventional materials, in one embodiment each of these components is made from a surgical stainless steel. In an alternative embodiment, the actuation member 22, the digit engagement member 30 and the retainer portion 40 are made of conventional polymeric materials. Although the ball bearings 46 may be made of other materials without departing from the scope of the present invention, in one embodiment the ball bearings are made of hardened steel. The actuation member 22 and the retainer portion 40 may be joined by any suitable conventional means, such as with screw fasteners or adhesives (not shown).

To operate the equipment 20 described above, the user grasps the equipment in a conventional fashion and engages a digit (e.g., a thumb) with the digit engagement member 30 so the digit is in contact with the engagement surface 32. In one embodiment, the user engages the digit engagement member 30 by inserting the digit in the aperture defined by the engagement surface 32. The user then operates the equipment 20 in a conventional fashion by moving the actuation member 22 relative to the body of the equipment. As the user moves the actuation member 22, the digit engagement member 30 rotates relative to the actuation member to reduce or eliminate relative motion between the user's digit and the engagement surface 32. In some preferred embodiments, the digit engagement surface 32 is adapted to be substantially free of sliding motion relative to the digit of the operator's hand as the actuation member 22 moves between the first position and the second position. As will be appreciated by those skilled in the art, the reducing relative motion between the user's digit and the actuation member 22 improves freedom of movement of the equipment and the user's hand, reduces opportunities for latex gloves to pinch the user's hand and reduces opportunities for the gloves to tear, thereby overcoming problems associated with conventional equipment described above. Moreover, the engagement surface 32 is less likely to wear the user's glove or hand because relative motion between hand and glove is significantly reduced.

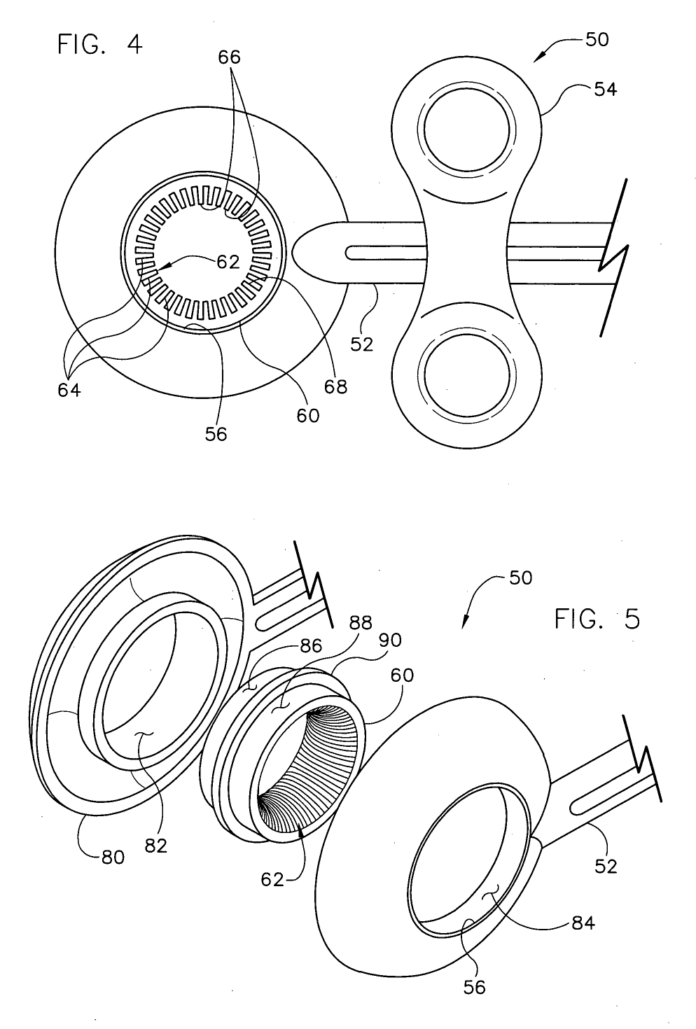

FIG. 4 illustrates a portion of equipment of a second embodiment of the present invention generally designated by the reference 50. The equipment 50 may be laparoscopic equipment. The equipment 50 includes at least one actuation member 52 adapted for movement between a first position and a second position. In some exemplary embodiments, the actuation member 52 slides relative to finger rings 54 (or another body forming part of the equipment) when the user actuates the equipment. The actuation member 22 includes an opening 56. In some embodiments, the opening 56 is positioned at an end of the actuation member 52 as shown. A digit engagement member 60 is rotatably mounted on the actuation member 52 inside the opening 56.

As shown in FIG. 5, the digit engagement member 60 has an engagement surface, generally designated 62, defining an aperture configured to receive a digit of an operator's hand during use. As shown in FIG. 4, the surface 62 includes a series of flexible leaves 64 spaced along surface around the aperture. Each of the leaves 64 has a tip 66 configured to engage a digit of an operator's hand during use. The leaves 64 deform to conform to the digit of the operator's hand, providing the digit with a snug but comfortable fit. Although the leaves 64 may be made of other materials without departing from the scope of the present invention, in one embodiment the leaves are formed from a flexible synthetic polymer that grips the user's digit so the digit engagement member 60 easily deforms to accept the digit, provides a snug fit and frictionally engages the user's digit during use. Although the leaves 64 may have other shapes without departing from the scope of the present invention, in one embodiment the leaves are tapered so each leaf is thinner at its tip 66 as shown in FIG. 5 and have rounded tips. Moreover, although the leaves 64 have other spacings without departing from the scope of the present invention, in one embodiment each pair of adjacent leaves 64 is spaced by a groove 68 having a uniform width. Further, in one embodiment, each of the grooves 68 separating adjacent leaves 64 has a substantially equal width compared to the other grooves.

As further illustrated in FIG. 5, the digit engagement member 60 is held in position by a separate retainer portion 80 fastened to the actuation member 52 adjacent the opening 56. Both the retainer portion 80 and the opening 56 in the actuation member 52 include a generally circular bushing surfaces 82, 84, respectively, which receive complementary bushing surfaces 86, 88, respectively, on the digit engagement member 60. Further, the digit engagement member 60 includes a circular rib 90 which engages a slot formed between the circular bushing surfaces 82, 84 of the retainer portion and actuation member 52 when assembled. Together, these surfaces form a bushing (or more broadly a bearing) which retains the digit engagement member 60 in the opening 56 of the actuation member 52 and allows it to rotate therein. Although the bushing surfaces 82, 84, 86, 88, the rib 90 and the slot surfaces may be made or coated from or with other low friction materials without departing from the scope of the present invention, in one embodiment at least one surface of each pair of complementary surfaces is coated with a silicone or polytetrafluoroethylene to reduce friction between the components. Although the actuation member 52 and the retainer portion 80 may be made over any suitable conventional materials, in one embodiment each of these components is made from a conventional polymeric material. The actuation member 52 and the retainer portion 80 may be joined by any suitable conventional means, such as with screw fasteners or adhesives (not shown).

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. Forceps for grasping tissue during a medical procedure, said forceps comprising:
a body;
a jaw pivotally mounted on the body for movement between a grasping position and a releasing position;
a lever operatively connected to the jaw to drive the jaw from the grasping position to the releasing position; and
a digit engagement member rotatably mounted on the lever, said digit engagement member having an engagement surface configured to engage a digit of an operator's hand during use, wherein said engagement surface rotates relative to the lever as the lever drives the jaw from the releasing position to the grasping position

2. Forceps as set forth in claim 1 wherein the engagement surface is adapted to be substantially free of sliding motion relative to the digit of the operator's hand as the lever drives the jaw from the releasing position to the grasping position.

3. Forceps as set forth in claim 1 further comprising a bearing connecting the digit engagement member to the lever to reduce friction between the digit engagement member and the lever.

4. Forceps as set forth in claim 3 wherein said bearing is a ball bearing.

5. Forceps as set forth in claim 1 wherein the lever includes an aperture and sized and shaped for receiving the digit engagement member.

6. Forceps as set forth in claim 5 wherein the lever aperture is generally circular and the digit engagement member is generally annular.

7. Forceps as set forth in claim 6 wherein at least one of the lever aperture and the digit engagement member is self lubricating.

8. Forceps as set forth in claim 1 wherein the engagement surface of the digit engagement member defines an aperture sized and shaped for receiving the digit of the operator's hand during use.

9. Forceps as set forth in claim 1 wherein the engagement surface of the digit engagement member is pliable to conform to the digit of the operator's hand during use.

10. Equipment manually operable by motion of a digit of an operator's hand, said equipment comprising:
a body;
an actuation member mounted on the body for movement between a first position and a second position; and
a digit engagement member rotatably mounted on the actuation member, said digit engagement member having an engagement surface configured to engage a digit of an operator's hand during use, wherein said engagement surface rotates relative to the actuation as the actuation member moves between the first position and the second position.

11. Equipment as set forth in claim 10 wherein the engagement surface is adapted to be substantially free of sliding motion relative to the digit of the operator's hand as the actuation member moves between the first position and the second position

12. Equipment as set forth in claim 10 further comprising a bearing connecting the digit engagement member to the actuation member to reduce friction between the digit engagement member and the actuation member.

13. Equipment as set forth in claim 12 wherein said bearing is a ball bearing.

14. Equipment as set forth in claim 10 wherein the actuation member includes an aperture and sized and shaped for receiving the digit engagement member.

15. Equipment as set forth in claim 14 wherein the actuation member aperture is generally circular and the digit engagement member is generally circular.

16. Equipment as set forth in claim 15 wherein at least one of the lever aperture and the digit engagement member is self lubricating.

17. Equipment as set forth in claim 10 wherein the engagement surface of the digit engagement member defines an aperture sized and shaped for receiving the digit of the operator's hand during use.

18. Equipment as set forth in claim 17 wherein the engagement surface of the digit engagement member is pliable to conform to the digit of the operator's hand during use.

19. Equipment manually operable by motion of a digit of an operator's hand, said equipment comprising:
a body;
an actuation member mounted on the body for movement between a first position and a second position; and
a digit engagement member mounted on the actuation member, said digit engagement member having an aperture defined by a series of flexible leaves spaced around the aperture, each of said leaves having a tip configured to engage a digit of an operator's hand during use, wherein said series of leaves deform to conform to the digit of the operator's hand.
